(19) United States
(12) Patent Application Publication
(10) Pub. No.: US 2016/0311739 A1
(43) Pub. Date: Oct. 27, 2016

(54) METHOD FOR PREPARING 1,6-HEXANEDIOL

(71) Applicant: BASF SE, Ludwigshafen (DE)

(72) Inventors: Christoph MÜLLER, Mannheim (DE); Martin BOCK, Ludwigshafen (DE); Marion DA SILVA, Mannheim (DE); Rolf-Hartmuth FISCHER, Heidelberg (DE); Benoit BLANK, Edingen-Neckarhausen (DE); Alois KINDLER, Grünstadt (DE); Johann-Peter MELDER, Böhl-Iggelheim (DE); Berhard OTTO, Schifferstadt (DE); Andreas HENNINGER, Laumersheim (DE)

(73) Assignee: BASF SE, Ludwigshafen (DE)

(21) Appl. No.: 15/103,483

(22) PCT Filed: Dec. 12, 2014
(86) PCT No.: PCT/EP2014/077630

§ 371 Date: Jun. 10, 2016

(30) Foreign Application Priority Data
Dec. 13, 2013 (EP) 13197156.6

(51) Int. Cl. C07C 29/149; C07C 31/20; C07C 51/36; C07C 29/80; C07C 29/17

(52) U.S. Cl. CPC C07C 29/149; C07C 29/80; C07C 29/177; C07C 51/36; C07C 31/20

(57) ABSTRACT

The invention relates to a process for preparing hexane-1,6-diol, in which
a) a muconic acid starting material is provided, selected from muconic acid, esters of muconic acid, lactones of muconic acid and mixtures thereof,
b) the muconic acid starting material is subjected to a reaction with hydrogen in the presence of at least one hydrogenation catalyst to hexane-1,6-diol, and
c) the output from the hydrogenation in step b) is subjected to a distillative separation to obtain hexane-1,6-diol.

### BACKGROUND OF THE INVENTION

The present invention relates to a process for preparing hexane-1,6-diol by subjecting muconic acid and/or one of its esters and/or one of its lactones to a hydrogenation of the double bonds and reduction of the carboxylic acid and/or carboxylic ester groups. The present invention further relates to hexane-1,6-diol preparable by means of this process.

### STATE OF THE ART

Hexane-1,6-diol is a sought-after monomer unit which is used predominantly in the polyester and polyurethane sector.

According to H.-J. Arpe, Industrielle Organische Chemie [Industrial Organic Chemistry], 6th edition (2007), Wiley-VCH-Verlag, pages 267 and 270, hexane-1,6-diol can be prepared by hydrogenation of adipic acid or adipic diesters in the presence of Cu catalysts, Co catalysts or Mn catalysts. The synthesis is effected at a temperature of 170 to 240° C. and a pressure of 5 to 30 MPa. Hexane-1,6-diol can also be obtained by catalytic hydrogenation of caprolactone.

EP 883 590 B1 discloses the use of a carboxylic acid mixture (DCS) rather than pure adipic acid or adipic esters prepared from pure adipic acid. This is obtained as a by-product in the oxidation of cyclohexane with oxygen or oxygen-comprising gases and by water extraction of the reaction mixture. The extract comprises adipic acid and 6-hydroxycaproic acid as main products, and additionally a multitude of mono- and dicarboxylic acids. The carboxylic acids are esterified with a lower alcohol. Adipic diesters are separated by distillation from the esterification mixture and hydrogenated catalytically to hexane-1,6-diol.

It is advantageous in this context that the DCS waste product is very inexpensive compared to pure adipic acid. On the other hand, a considerable level of distillation complexity is necessary to produce pure hexane-1,6-diol. Particular difficulties are presented by the distillative removal of the cyclohexane-1,4-diols which occur as by-products.

WO 2010/115759 describes a process for preparing hexane-1,6-diol by catalytic hydrogenation of ester mixtures which comprise oligo- and polyesters of adipic acid and 6-hydroxycaproic acid as main components and are obtained by esterification of DCS with diols, especially hexane-1,6-diol or diol mixtures.

Adipic acid is conventionally synthesized by oxidation of cyclohexene or cyclohexanone proceeding from benzene. It can also be obtained in an environmentally friendly manner from biogenic sources.

WO 2012/141993 A1 describes the preparation of hexamethylenediamine (HMDA) from muconic diesters, wherein the muconic diesters are amidated in a first step and then reduced directly to HMDA (route 1) or, after the amidation, are dehydrated to give nitriles and then hydrogenated to give HMDA (route 2) or, after the amidation, are hydrogenated to give adipamide, dehydrated to give adiponitrile and then hydrogenated to give HMDA (route 3). According to the teaching of this document, hexane-1,6-diol occurs neither as intermediate nor as end product.

U.S. Pat. No. 4,968,612 describes a fermentation process for preparation of muconic acid and the hydrogenation of the muconic acid thus obtained to adipic acid. Specifically, muconic acid is reacted as a 40% by weight slurry in acetic acid and in the presence of a palladium catalyst on charcoal. The water content of the acetic acid used is unspecified. A disadvantage of this mode of reaction is the use of corrosive acetic acid, which entails the use of high-quality corrosion-resistant reactors.

K. M. Draths and J. W. Frost, J. Am. Chem. Soc. 1994, 116, 399-400 and W. Niu et al., Biotechnol. Prog. 2002, 18, 201-211 describe the preparation of cis,cis-muconic acid from glucose by biocatalyzed synthesis with subsequent hydrogenation of the cis,cis-muconic acid with the aid of a platinum catalyst to adipic acid. In the two cases, the pH of the fermentation mixture prior to the hydrogenation is adjusted to above 6.3, or to a value of 7.0. This results in a solution of muconic salts. Since, in the two cases, the fermentation broth is first centrifuged and only the supernatant is used for hydrogenation, and according to the procedure of Niu et al. the supernatant is additionally twice admixed with activated carbon and filtered prior to the hydrogenation, it can be assumed that the hydrogenation mixture does not comprise any solid muconic acid.

A further process for preparing muconic acid from renewable sources is described, for example, in WO 2010/148080 A2. According to example 4, in paragraphs [0065] and [0066] of this document, 15 g of cis,cis-muconic acid and 150 ml of water are heated under water reflux for 15 minutes. After cooling to room temperature, filtration and drying, 10.4 g (69%) of cis,trans-muconic acid are obtained. The mother liquor (4.2 g=28% by weight, based on cis,cis-muconic acid) no longer consists of muconic acid. It comprises lactones and further, unknown reaction products.

J. M. Thomas et al., Chem. Commun. 2003, 1126-1127, describe the hydrogenation of muconic acid to adipic acid with the aid of bimetallic nanocatalysts which have been intercalated into the pores of a mesoporous silicon dioxide by means of specific anchor groups, in pure ethanol.

J. A. Elvidge et al., J. Chem. Soc. 1950, 2235-2241, describe the preparation of cis,trans-muconic acid and the hydrogenation thereof to adipic acid in ethanol in the presence of a platinum catalyst. No details are given of the amount of solvent used and the catalyst.

X. She et al., ChemSusChem 2011, 4, 1071-1073, describe the hydrogenation of trans,trans-muconic acid to adipic acid with rhenium catalysts on a titanium dioxide support in solvents selected from methanol, ethanol, 1-butanol, acetone, toluene and water. The hydrogenations are performed exclusively at an elevated temperature of 120° C. With the catalyst used, only a low selectivity based on the adipic acid is achieved; the main product is dihydromuconic acid.

WO 2010/141499 describes the oxidation of lignin to vanillic acid, the decarboxylation of the latter to 2-methoxyphenol and further conversion to catechol, and finally oxidation to muconic acid, and hydrogenation of muconic acid obtained in this way with various transition metal catalysts to adipic acid. The solvent used for the hydrogenation is unspecified.

It is an object of the present invention to provide an economically viable process for preparing hexane-1,6-diol. More particularly, this process is not to proceed from petrochemical C₆ starting materials, but from C₆ starting materials preparable from renewable raw materials. At the same time, hexane-1,6-diol is to be made available in high yield and purity.

It has now been found that, surprisingly, this object is achieved by subjecting a muconic acid starting material selected from muconic acid, esters of muconic acid, lactones of muconic acid and mixtures thereof to a one- or two-stage conversion with hydrogen to hydrogenate the double bonds and reduce the carboxylic acid and/or carboxylic ester groups to hexane-1,6-diol. More particularly, the muconic acid used originates from renewable (biogenic) sources.

### SUMMARY OF THE INVENTION

The invention firstly provides a process for preparing hexane-1,6-diol, in which
a) a muconic acid starting material is provided, selected from muconic acid, esters of muconic acid, lactones of muconic acid and mixtures thereof,
b) the muconic acid starting material is subjected to a reaction with hydrogen in the presence of at least one hydrogenation catalyst to hexane-1,6-diol, and
c) the output from the hydrogenation in step b) is subjected to a distillative separation to obtain hexane-1,6-diol.

The invention further provides for the use of a poly(muconic ester) of the general formula (VI)

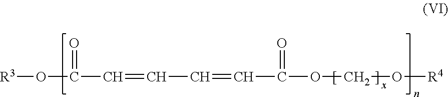

in which
x is an integer from 2 to 6,
n is an integer from 1 to 100,
R³ is H, straight-chain or branched C₁-C₅-alkyl or a HO—(CH₂)ₓ— group,
R⁴ is H or a —C(═O)—CH═CH—CH═CH—COOR⁵ group in which R⁵ is H or straight-chain or branched C₁-C₅-alkyl,

with the proviso that, when n=1, either R³ is H and R⁴ is —C(═O)—CH═CH—CH═CH—COOR⁵ or R³ is a HO—(CH₂)ₓ— group and R⁴ is H,

for preparation of hexane-1,6-diol.

The invention further provides hexane-1,6-diol having a C¹⁴/C¹² isotope ratio in the range from 0.5×10⁻¹² to 5×10⁻¹².

The invention further provides hexane-1,6-diol preparable proceeding from muconic acid synthesized biocatalytically from at least one renewable raw material.

Specifically, the muconic acid starting material provided in step a) does not comprise any salts of muconic acid.

In a specific embodiment, the hydrogenation in step b) is effected in the liquid phase in the presence of water as the sole solvent.

In a very specific embodiment, the hydrogenation in step b) is effected in the liquid phase in the presence of water as the sole solvent, wherein the hydrogenation catalyst used is a heterogeneous transition metal catalyst.

In an even more specific embodiment, the hydrogen in step b) comprises the following component steps:
b1) hydrogenating muconic acid or one of its esters in water as the sole solvent to adipic acid in the presence of a first heterogeneous hydrogenation catalyst, and
b2) hydrogenating the adipic acid obtained in step b1) in water as the sole solvent to hexane-1,6-diol in the presence of a second heterogeneous hydrogenation catalyst,

the hydrogenation preferably being effected continuously at least in step b2).

Muconic acid (hexadiene-2,4-dicarboxylic acid) exists in three stereoisomeric forms, the cis,cis form, the cis,trans form and the trans,trans form, which may be present as a mixture. All three forms are crystalline compounds having high melting points (decomposition); see, for example, Römpp Chemie Lexikon, 9th edition, volume 4, page 2867. It has been found that hydrogenation of muconic acid melts is barely possible by industrial means, since the very particularly preferred hydrogenation temperatures are well below the melting points. Therefore, an inert solvent having maximum solubility for muconic acid would be desirable for the hydrogenation. At first glance, water appears unsuitable to the person skilled in the art as a solvent, since muconic acid, in contrast to adipic acid, is sparingly soluble within the temperature range from 20 to 100° C. As described above, WO 2010/148080 teaches that cis,trans-muconic acid is obtained in only 69% yield when cis,cis-muconic acid is heated in water under reflux with subsequent crystallization. The remaining mother liquor no longer consists of muconic acid, but comprises lactones and further, unknown reaction products. On the basis of these results, the person skilled in the art, in the hydrogenation of muconic acid suspended in water, in accordance with the preferred embodiment of the present invention, would have expected much lower adipic acid yields.

### EMBODIMENTS OF THE INVENTION

Specifically, the invention encompasses the following preferred embodiments:
1. A process for preparing hexane-1,6-diol, in which
   a) a muconic acid starting material is provided, selected from muconic acid, esters of muconic acid, lactones of muconic acid and mixtures thereof,
   b) the muconic acid starting material is subjected to a reaction with hydrogen in the presence of at least one hydrogenation catalyst to hexane-1,6-diol, and
   c) the output from the hydrogenation in step b) is subjected to a distillative separation to obtain hexane-1,6-diol.
2. The process according to embodiment 1, wherein a muconic acid starting material is provided in step a), in which the muconic acid originates from a renewable source, and is preferably prepared by biocatalytic synthesis from at least one renewable raw material.
3. The process according to embodiment 1 or 2, wherein the muconic acid used in step a) has a ¹⁴C-to-¹²C isotope ratio in the range from 0.5×10⁻¹² to 5×10⁻¹².
4. The process according to any of embodiments 1 to 3, wherein the hydrogenation in step b) is effected using a muconic acid starting material selected from muconic acid, muconic monoesters, muconic diesters, poly(muconic esters) and mixtures thereof.
5. The process according to any of embodiments 1 to 3, wherein the hydrogenation in step b) is effected using a muconic acid starting material selected from the lactones (III), (IV) and (V) and mixtures thereof:

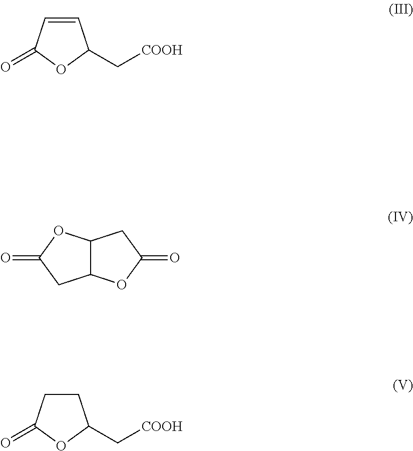 [SMILES: O=C(O)CC1C=CC(=O)O1 ; O=C1CC2OC(=O)CC2O1 ; O=C(O)CC1CCC(=O)O1]

6. The process according to any of embodiments 1 to 5, wherein the hydrogenation in step b) is effected in the liquid phase in the presence of a solvent selected from water, aliphatic C₁ to C₅ alcohols, aliphatic C₂ to C₆ diols, ethers and mixtures thereof.
7. The process according to any of embodiments 1 to 5, wherein the hydrogenation in step b) is effected in the liquid phase in the presence of water as the sole solvent.
8. The process according to any of embodiments 1 to 5, wherein the hydrogenation in step b) is effected in the gas phase using for the hydrogenation a muconic diester selected from compounds of the general formula (II):

R¹OOC—CH═CH—CH═CH—COOR² (II)
in which the R¹ and R² radicals are each independently straight-chain or branched C₁-C₅-alkyl.
9. The process according to any of the preceding embodiments, wherein the hydrogenation catalyst used in step b) is a homogeneous or heterogeneous transition metal catalyst, preferably a heterogeneous transition metal catalyst.
10. The process according to any of embodiments 1 to 9, wherein in step b) a muconic acid starting material is used, selected from muconic acid, muconic monoesters and mixtures thereof, and the hydrogenation catalyst comprises at least 50% by weight of cobalt, ruthenium or rhenium, based on the total weight of the reduced catalyst.
11. The process according to any of embodiments 1 to 9, wherein in step b) a muconic acid starting material is used, selected from muconic diesters, poly(muconic esters) and mixtures thereof, and the hydrogenation catalyst Hc1) comprises at least 50% by weight of copper, based on the total weight of the reduced catalyst.
12. The process according to any of the preceding embodiments, wherein the hydrogenation in step b) is effected at a temperature within the range from 50 to 300° C.
13. The process according to any of the preceding embodiments, wherein the hydrogenation in step b) is effected at a partial hydrogen pressure within a range from 100 to 300 bar.
14. The process according to any of embodiments 1 to 13, wherein the hydrogenation in step b) is effected without intermediate isolation of adipic acid or any ester of adipic acid.
15. The process according to any of embodiments 1 to 13, wherein step b) comprises the following component steps:
   b1) hydrogenating muconic acid or one of its esters in aqueous solution to adipic acid in the presence of a first hydrogenation catalyst, and
   b2) hydrogenating the adipic acid in aqueous solution to hexane-1,6-diol in the presence of a second hydrogenation catalyst.
16. The process according to embodiment 15, wherein the first hydrogenation catalyst is Raney cobalt and/or Raney nickel.
17. The process according to embodiment 15 or 16, wherein the second catalyst, based on the total weight of the reduced catalyst, comprises at least 50% by weight of elements selected from the group consisting of rhenium, iron, ruthenium, cobalt, rhodium, iridium, nickel and copper.
18. The process according to any of the embodiments 15 to 17, wherein the hydrogenation in step b1) is effected at a temperature within the range from 50 to 160° C. and the hydrogenation in step b2) is effected at a temperature within the range from 160 to 240° C.
19. The process according to any of embodiments 15 to 18, wherein adipic acid-containing water which is obtained in the isolation of the second catalyst on completion of step b2) is used as solvent in step b1).
20. The process according to any of the preceding embodiments, wherein the hydrogenation is conducted in n series-connected hydrogenation reactors, where n is an integer of at least two.
21. The process according to embodiment 20, wherein the 1st to (n-1)th reactor has a stream from the reaction zone conducted within an external circuit.
22. The process according to either of embodiments 20 and 21, wherein the hydrogenation is conducted adiabatically in the nth reactor.
23. The use of a poly(muconic ester) of the general formula (VI)

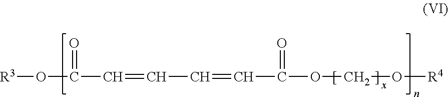

in which
x is an integer from 2 to 6,
n is an integer from 1 to 100,
R³ is H, straight-chain or branched C₁-C₅-alkyl or a HO—(CH₂)ₓ— group,
R⁴ is H or a —C(═O)—CH⊚CH—CH═CH—COOR⁵ group in which R⁵ is H or straight-chain or branched C₁-C₅-alkyl,
with the proviso that, when n=1, either R³ is H and R⁴ is —C(═O)—CH═CH—CH═CH—COOR⁵ or R³ is a HO—(CH₂)ₓ— group and R⁴ is H,
for preparation of hexane-1,6-diol.
24. Hexane-1,6-diol having a C¹⁴/C¹² isotope ratio in the range from 0.5×10⁻¹² to 5×10⁻¹².
25. Hexane-1,6-diol preparable proceeding from muconic acid synthesized biocatalytically from at least one renewable raw material.

### DETAILED DESCRIPTION OF THE INVENTION

In the context of the present invention, esters of muconic acid refer to the esters with a separate (external) alcohol component. Lactones of muconic acid are understood to mean the compounds (III) and (IV) obtainable by intramolecular Michael addition, and the product (V) of the hydrogenation of the compound (III):

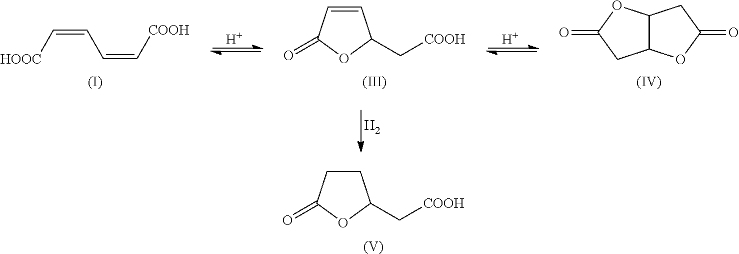

The lactone (V) can also form through intramolecular Michael addition from dihydromuconic acid. Irrespective of its preparation, the lactone (V) can be referred to in a formal sense as “hydrogenated monolactone of muconic acid”. In the context of the invention, the lactone (V) is also regarded as the lactone of muconic acid.

The muconic acid provided in step a) of the process according to the invention originates from renewable sources. In the context of the invention, this is understood to mean natural (biogenic) sources and not fossil sources such as mineral oil, natural gas and coal. Preferably, the muconic acid provided in step a) of the process according to the invention originates from carbohydrates, e.g. starch, cellulose and sugars, or from lignin. Compounds obtained from renewable sources, for example muconic acid, have a different ¹⁴C-to-¹²C isotope ratio than compounds obtained from fossil sources such as mineral oil. The muconic acid used in step a) accordingly preferably has a ¹⁴C-to-¹²C isotope ratio in the range from 0.5×10⁻¹² to 5×10⁻¹².

The preparation of muconic acid from renewable sources can be effected by all processes known to those skilled in the art, preferably by biocatalytic means. The biocatalytic preparation of muconic acid from at least one renewable raw material is described, for example, in the following documents: U.S. Pat. No. 4,968,612, WO 2010/148063 A2, WO 2010/148080 A2, and also K. M. Draths and J. W. Frost, J. Am. Chem. Soc. 1994, 116, 339-400 and W. Niu et al., Biotechnol. Prog. 2002, 18, 201-211.

As explained above, muconic acid (hexadiene-2,4-dicarboxylic acid) exists in three isomeric forms, the cis,cis form, the cis,trans form and the trans,trans form, which may be present as a mixture. The term “muconic acid” in the context of the invention encompasses the different conformers of muconic acid in any composition. Suitable feedstocks for the reaction with hydrogen in step b) of the process according to the invention are in principle all conformers of muconic acid and/or esters thereof and any mixtures thereof.

In a preferred embodiment, in step b) of the process according to the invention, a feedstock enriched in cis,trans-muconic acid and/or esters thereof or consisting of cis,trans-muconic acid and/or esters thereof is provided. This is because cis,trans-muconic acid and esters thereof have a higher solubility in water and in organic media than cis,cis-muconic acid and trans,trans-muconic acid.

If, in step b) of the process according to the invention, a feedstock comprising at least one component selected from cis,cis-muconic acid, trans,trans-muconic acid and/or esters thereof is provided, it is subjected, before or during the hydrogenation to an isomerization to cis,trans-muconic acid or esters thereof. The isomerization of cis,cis-muconic acid to cis,trans-muconic acid is depicted in the following scheme:

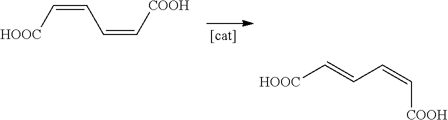 [SMILES: O=C(O)/C=C\C=C/C(=O)O ; O=C(O)/C=C\C=C\C(=O)O]

Useful catalysts are especially inorganic or organic acids, hydrogenation catalysts, iodine or UV radiation. Suitable hydrogenation catalysts are described hereinafter. The isomerization can be effected, for example, by the process described in WO 2011/085311 A1.

Preferably, the feedstock for the reaction with hydrogen in step b) consists to an extent of at least 80% by weight, more preferably at least 90% by weight, of cis,trans-muconic acid and/or esters thereof, based on the total weight of all the muconic acid and muconic ester conformers present in the feedstock.

For the hydrogenation in step b), preference is given to using a muconic acid starting material selected from muconic acid, muconic monoesters, muconic diesters, poly(muconic esters), lactones of muconic acid and mixtures thereof. In the context of the present invention, the term “muconic polyester” also refers to oligomeric muconic esters having at least one repeat unit derived from muconic acid or the dial used to form the ester, and at least two complementary repeat units bonded via carboxylic ester groups.

Preferably, the muconic monoester used is at east one compound of the general formula (I)

R¹OOC—CH═CH—CH═CH—COOH (I)

in which the R¹ radicals are each independently straight-chain or branched C₁-C₅-alkyl.

Preferably, the muconic diester used is at least one compound of the general formula (II)

R¹OOC—CH═CH—CH═CH—COOR² (II)

in which the R¹ and R² radicals are each independently straight-chain or branched C₁-C₅-alkyl.

Preferably, the poly(muconic ester) used is at least one compound of the general formula (VI)

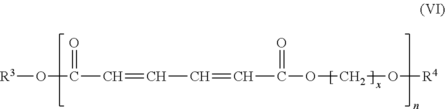

in which
x is an integer from 2 to 6,
n is an integer from 1 to 100,
R³ is H, straight-chain or branched C₁-C₅-alkyl or a HO—(CH₂)ₓ— group,
R⁴ is H or a —C(═O)—CH═CH—CH═CH—COOR⁵ group in which R⁵ is H or straight-chain or branched C₁-C₅-alkyl,

with the proviso that, when n=1, either R³ is H and R⁴ is —C(═O)—CH═CH—CH═CH—COOR⁵ or R³ is a HO—(CH₂)ₓ— group and R⁴ is H.

In the context of the invention, the degree of polymerization of the poly(muconic ester) refers to the sum total of repeat units derived in a formal sense from muconic acid and of the repeat units derived in a formal sense from dials HO—(CH₂)ₓ—OH.

In a first preferred embodiment, the hydrogenation in step b) is effected using a muconic acid starting material selected from muconic acid, muconic monoesters, muconic diesters, poly(muconic esters) and mixtures thereof.

In a second preferred embodiment, the hydrogenation in step b) is effected using a muconic acid starting material selected from the lactones (III), (IV) and (V) and mixtures thereof:

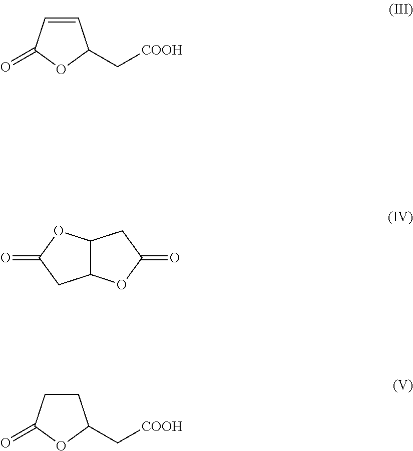 [SMILES: O=C(O)CC1C=CC(=O)O1 ; O=C1CC2OC(=O)CC2O1 ; O=C(O)CC1CCC(=O)O1]

Specifically, the hydrogenation in step b) is effected using a muconic acid starting material selected from muconic acid, muconic monoesters, muconic diesters, poly(muconic esters) and mixtures thereof, wherein the hydrogenation is effected in the liquid phase.

In a first embodiment of the process according to the invention, the hydrogenation in step b) is effected in the liquid phase in the presence of a solvent selected from water, aliphatic C₁ to C₅ alcohols, aliphatic C₂ to C₆ diols, ethers and mixtures thereof. Preferably, the solvent is selected from water, methanol, ethanol, n-propanol, isopropanol, n-butanol, sec-butanol, isobutanol and tert-butanol, ethylene glycol, propane-1,3-diol, butane-1,4-diol, pentane-1,5-diol, hexane-1,6-diol, tetrahydrofuran, 2-methyltetrahydrofuran, diethyl ether, methyl tert-butyl ether and mixtures thereof. Preference is given to aliphatic C₁ to C₅ alcohols, water and mixtures of these solvents. Particular preference is given to methanol, n-butanol, isobutanol, water and mixtures of these solvents. It is additionally preferable to use the hexane-1,6-dial target product as the solvent. In this case, hexane-1,6-diol can be used alone or in a mixture with alcohols and/or water.

It is preferable that, for the hydrogenation in the liquid phase, a solution comprising 10 to 60% by weight of muconic acid or one of its esters, more preferably 20 to 50% by weight, most preferably 30 to 50% by weight, is used.

In a second preferred embodiment, the hydrogenation in step b) is effected using at least muconic diester of the general formula (II)

R¹OOC—CH═CH—CH═CH—COOR² (II)

in which the R¹ and R² radicals are each independently straight-chain or branched C₁-C₅-alkyl, and wherein the hydrogenation is effected in the gas phase.

Hydrogenation catalysts suitable for the reaction in step b) are in principle the transition metal catalysts known to the person skilled in the art for hydrogenation of carbon-carbon double bonds. In general, the catalyst comprises at least one transition metal of groups 7, 8, 9, 10 and 11 of the IUPAC Periodic Table. Preferably, the catalyst has at least one transition metal from the group of Mn, Re, Fe, Ru, Co, Rh, Ir, Ni, Pd, Pt, Cu and Au. More preferably, the catalyst has at least one transition metal from the group of Co, Ni, Cu, Re, Fe, Ru, Rh, Ir. The hydrogenation catalysts consist of the transition metals mentioned as such or comprise the transition metals mentioned applied to a support, as precipitated catalysts, as Raney catalysts or as mixtures thereof.

Inert support materials used for the hydrogenation catalysts used in accordance with the invention in step b) may be virtually all prior art support materials as used advantageously in the production of supported catalysts, for example carbon, SiO₂ (quartz), porcelain, magnesium oxide, tin dioxide, silicon carbide, TiO₂ (rutile, anatase), Al₂O₃ (alumina), aluminum silicate, steatite (magnesium silicate), zirconium silicate, cerium silicate or mixtures of these support materials. Preferred support materials are carbon, aluminum oxide and silicon dioxide. A particularly preferred support material is carbon. The silicon dioxide support materials used for catalyst production may be silicon dioxide materials of different origin and production, for example fumed silicas or silicas produced by wet-chemical means, such as silica gels, aerogels or precipitated silicas (for production of the various SiO₂ starting materials see: W. Büchner, R. Schliebs, G. Winter, K. H. Büchel: Industrielle Anorganische Chemie [Industrial Inorganic Chemistry], 2nd ed., p. 532-533, VCH Verlagsgesellschaft, Weinheim 1986).

The hydrogenation catalysts can be used in the form of shaped bodies, for example in the form of spheres, rings, cylinders, tubes, cuboids or other geometric bodies. Unsupported catalysts can be shaped by customary methods, for example by extrusion, tableting etc. The shape of supported catalysts is determined by the shape of the support. Alternatively, the support can be subjected to a shaping process before or after the application of the catalytically active component(s). The hydrogenation catalysts can be used, for example, in the form of pressed cylinders, tablets, pellets, wagonwheels, rings, stars, or extrudates such as solid extrudates, polylobal extrudates, hollow extrudates and honeycombs, or other geometric bodies.

The catalyst particles generally have a mean (greatest) diameter of 0.5 to 20 mm, preferably 1 to 10 mm. These include, for example, transition metal catalysts K in the form of tablets, for example having a diameter of 1 to 7 mm, preferably 2 to 6 mm, and a height of 3 to 5 mm, rings having external diameter, for example, 4 to 7 mm, preferably 5 to 7 mm, height 2 to 5 mm and hole diameter 2 to 3 mm, or extrudates of different length with diameter, for example, 1.0 to 5 mm. Shapes of this kind can be obtained in a manner known per se by tableting or extrusion. For this purpose, it is possible to add customary auxiliaries to the catalyst composition, for example lubricants such as graphite, polyethylene oxide, cellulose or fatty acids (such as stearic acid) and/or shaping auxiliaries and reinforcing agents, such as fibers of glass, asbestos or silicon carbide.

The catalyst may also be present in the form either of a homogeneous or heterogeneous catalyst under the hydrogenation conditions. Preferably, the catalyst is in the form of a heterogeneous catalyst under the hydrogenation conditions. If a heterogeneous catalyst is used, it may be applied, for example, to a support in mesh form. Alternatively or additionally, the heterogeneous catalyst may be applied to the inner wall of a tubular support, in which case the reaction mixture flows through the tubular support. Alternatively or additionally, the catalyst can be used in the form of a particulate solid. In a preferred embodiment, the hydrogenation in step b) is effected in the liquid phase, and the catalyst is in the form of a suspension. If a liquid reaction output is removed from the reaction zone, the suspended catalyst can be kept in the reaction zone by retention methods known to those skilled in the art. These retention methods preferably include crossflow filtration, gravitational filtration and/or filtration by means of at least one filter cartridge.

Hydrogenation of Muconic Acid, Muconic Monoesters and Lactones

In a first process variant, the hydrogenation in step b) is effected using a muconic acid starting material selected from muconic acid, muconic monoesters, lactones of muconic acid and mixtures thereof.

In this process variant, the hydrogenation in step b) is preferably effected using a hydrogenation catalyst comprising at least 50% by weight of cobalt, ruthenium or rhenium, based on the total weight of the reduced catalyst.

If the hydrogenation is effected using catalysts comprising at least 50% by weight of cobalt, the latter may further comprise especially phosphoric acid and/or further transition metals, preferably copper, manganese and/or molybdenum.

The preparation of a suitable catalyst precursor is known from DE 2321101. This comprises, in the unreduced, calcined state, 40 to 60% by weight of cobalt (calculated as Co), 13 to 17% by weight of copper (calculated as Cu), 3 to 8% by weight of manganese (calculated as Mn), 0.1 to 5% by weight of phosphates (calculated as H₃PO₄) and 0.5 to 5% by weight of molybdenum (calculated as MoO₃). EP 636 409 B1 describes the preparation of further suitable cobalt catalyst precursors consisting to an extent of 55 to 98% by weight of cobalt, to an extent of 0.2 to 15% by weight of phosphorus, to an extent of 0.2 to 15% by weight of manganese and to an extent of 0.2 to 15% by weight of alkali metals (calculated as oxide). Catalyst precursors of this kind can be reduced to the active catalysts comprising metallic cobalt by treatment with hydrogen or mixtures of hydrogen and the inert gases such as nitrogen. These catalysts are unsupported catalysts consisting very predominantly of metal and not comprising any catalyst support.

Hydrogenation of Muconic Diesters and Muconic Polyesters

In a first process variant, the hydrogenation in step b) is effected using a muconic acid starting material selected from muconic diesters, poly(muconic esters) and mixtures thereof.

In this process variant, the hydrogenation in step b) is preferably effected using a hydrogenation catalyst comprising at least 50% by weight of copper, based on the total weight of the reduced catalyst.

Useful catalysts are in principle all homogeneous and heterogeneous catalysts suitable for hydrogenation of carbonyl groups, such as metals, metal oxides, metal compounds or mixtures thereof. Examples of homogeneous catalysts are described, for example, in Houben-Weyl, Methoden der Organischen Chemie [Methods of Organic Chemistry], volume IV/1c, Georg Thieme Verlag Stuttgart, 1980, p. 45-67, and examples of heterogeneous catalysts are described, for example, in Houben-Weyl, Methoden der Organischen Chemie, volume IV/1c, p. 16 to 26.

Preference is given to using catalysts comprising one or more elements from transition groups I and VI to VIII of the Periodic Table of the Elements, preferably copper, chromium, molybdenum, manganese, rhenium, ruthenium, cobalt, nickel or palladium, more preferably copper, cobalt or rhenium.

In the hydrogenation of the muconic diesters, oligoesters and polyesters too, it is possible to use the cobalt-, ruthenium- or rhenium-comprising catalysts already mentioned. It is preferable, however, rather than these catalysts, to use catalysts comprising at least 50% by weight of copper (based on the total weight of the reduced catalyst).

The catalysts may consist solely of active components, or the active components thereof may be applied to supports. Suitable support materials are especially Cr2O3, Al2O3, SiO2, ZrO2, ZnO, BaO and MgO or mixtures thereof.

Particular preference is given to catalysts as described in EP 0 552 463 A1. These are catalysts which, in the oxidic form, have the composition

CuaAlbZrcMndOx

where a>0, b>0, c≧0, d>0, a>b/2, b>a/4, a>c and a>d, and x denotes the proportion of oxygen ions required per formula unit to give electronic neutrality. These catalysts can be prepared, for example, according to the specifications of EP 552 463 A1, by precipitation of sparingly soluble compounds from solutions comprising the corresponding metal ions in the form of salts thereof. Suitable salts are, for example, halides, sulfates and nitrates. Suitable precipitants are all agents which lead to the formation of those insoluble intermediates that can be converted to the oxides by thermal treatment. Particularly suitable intermediates are hydroxides and carbonates or hydrogencarbonates, and so alkali metal carbonates or ammonium carbonate are used as particularly preferred precipitants. Thermal treatment of the intermediates is effected at temperatures in the range from 500° C. to 1000° C. The BET surface area of such catalysts is between 10 and 150 m2/g.

Additionally suitable are catalysts which have a BET surface area of 50 to 120 m2/g, fully or partly comprise crystals having spinel structure, and comprise copper in the form of copper oxide.

WO 2004/085 356 A1 also describes copper catalysts suitable for the process according to the invention, which comprise copper oxide, aluminum oxide and at least one of the oxides of lanthanum, tungsten, molybdenum, titanium or zirconium, and additionally pulverulent metallic copper, copper flakes, pulverulent cement, graphite or a mixture thereof. These catalysts are particularly suitable for all the ester hydrogenations mentioned.

The hydrogenation in step b) can be conducted batchwise or continuously, preference being given to a continuous hydrogenation.

The catalyst hourly space velocity in continuous mode is preferably 0.1 to 2 kg, more preferably 0.5 to 1 kg, of starting material to be hydrogenated per kg of hydrogenation catalyst and hour.

The molar ratio of hydrogen to muconic acid starting material is preferably 50:1 to 10:1, more preferably 30:1 to 20:1. This muconic acid starting material is selected in accordance with the invention from muconic acid, esters of muconic acid, lactones of muconic acid and mixtures thereof.

If the hydrogenation in step b) is effected using a muconic acid starting material selected from at least two of the aforementioned compounds, the amount of hydrogen used is selected as a function of the proportion of the compounds to be hydrogenated according to the aforementioned assessment rule.

In a specific execution of the process according to the invention, the hydrogenation is effected in n series-connected hydrogenation reactors, where n is an integer of at least 2. Suitable values of n are 2, 3, 4, 5, 6, 7, 8, 9 and 10. Preferably, n is 3 to 6 and especially 2 or 3. In this execution, the hydrogenation is preferably effected continuously.

The reactors used for hydrogenation may each independently have one or more reaction zones within the reactor. The reactors may be identical or different reactors. These may, for example, each have the same or different mixing characteristics and/or be divided once or more than once by internals.

Suitable pressure-resistant reactors for the hydrogenation are known to those skilled in the art. These include the reactors generally customary for gas-liquid reactions, for example tubular reactors, shell and tube reactors, gas circulation reactors, bubble columns, loop apparatuses, stirred tanks (which may also be configured as stirred tank cascades), airlift reactors etc.

The process according to the invention using heterogeneous hydrogenation catalysts can be conducted in fixed bed mode or suspension mode. Operation in fixed bed mode can be conducted, for example, in liquid phase mode or in trickle mode. In this case, the hydrogenation catalysts are preferably used in the form of shaped bodies as described above, for example in the form of pressed cylinders, tablets, pellets, wagonwheels, rings, stars, or extrudates such as solid extrudates, polylobal extrudates, hollow extrudates, honeycombs etc.

In suspension mode, heterogeneous catalysts are likewise used. The heterogeneous catalysts are usually used in a finely divided state and are in fine suspension in the reaction medium.

Suitable heterogeneous catalysts and processes for preparation thereof have been described above.

In the case of hydrogenation over a fixed bed, a reactor with a fixed bed arranged in the interior thereof, through which the reaction medium flows, is used. This fixed bed may be formed from a single bed or from a plurality of beds. Each bed may have one or more zones, at least one of the zones comprising a material active as a hydrogenation catalyst. Each zone may have one or more different catalytically active materials and/or one or more different inert materials. Different zones may each have identical or different compositions. It is also possible to provide a plurality of catalytically active zones separated from one another, for example, by inert beds. The individual zones may also have different catalytic activity. To this end, it is possible to use different catalytically active materials and/or to add an inert material at least to one of the zones. The reaction medium which flows through the fixed bed comprises at least one liquid phase. The reaction medium may also additionally comprise a gaseous phase.

The reactors used in the hydrogenation in suspension are especially loop apparatuses such as jet loops or propeller loops, stirred tanks, which may also be configured as stirred tank cascades, bubble columns or airlift reactors.

Preferably, the continuous hydrogenation of the process according to the invention is effected in at least two series-connected fixed bed reactors. The reactors are preferably operated in cocurrent. The feed streams can be fed in either from the top or from the bottom.

If desired, in a hydrogenation apparatus composed of n reactors, at least two of the reactors (i.e. 2 to n of the reactors) may have different temperatures. In a specific embodiment, every downstream reactor is operated with a higher temperature than the previous reactor. In addition, each of the reactors may have two or more reaction zones with different temperatures. For example, a different temperature, preferably a higher temperature, can be established in a second reaction zone than in the first reaction zone, or a higher temperature than in an upstream reaction zone can be established in every downstream reaction zone, for example in order to achieve substantially full conversion in the hydrogenation.

In a specific embodiment, the hydrogenation in step b) is effected using a hydrogenation apparatus composed of at least 2 reactors or at least one reactor having at least two reaction zones. In that case, the hydrogenation is effected first within a temperature range from 50 to 160° C. and then within a temperature range from 160 to 240° C. In this procedure, essentially the carbon-carbon double bonds can first be hydrogenated in the upstream part of the hydrogenation apparatus, and then essentially the carboxylic acid and/or carboxylic ester groups can be reduced in the downstream part of the hydrogenation apparatus.

If desired, in a hydrogenation apparatus composed of n reactors, at least two of the reactors (i.e. 2 to n of the reactors) may have different pressures. In a specific embodiment, every downstream reactor is operated with a higher pressure than the previous reactor.

The hydrogen required for the hydrogenation can be fed into the first and optionally additionally into at least one further reactor. Preferably, hydrogen is fed only into the first reactor. The amount of hydrogen fed to the reactors is calculated from the amount of hydrogen consumed in the hydrogenation reaction and any amount of hydrogen discharged with the offgas.

The proportion of compound to be hydrogenated which has been converted in the particular reactor can be adjusted, for example, via the reactor volume and/or the residence time in the reactor.

The conversion in the first reactor, based on the adipic acid or adipic ester formed, is preferably at least 70%, more preferably at least 80%.

The overall conversion in the hydrogenation, based on hydrogenatable starting material, is preferably at least 97%, more preferably at least 98%, especially at least 99%.

The selectivity in the hydrogenation, based on hexane-1,6-diol formed, is preferably at least 97%, more preferably at least 98%, especially at least 99%.

To remove the heat of reaction which arises in the exothermic hydrogenation, it is possible to provide one or more of the reactors with at least one cooling apparatus. In a specific embodiment, at least the first reactor is provided with a cooling apparatus. The heat of reaction can be removed by cooling of an external circulation stream or by internal cooling in at least one of the reactors. For the internal cooling, it is possible to use the apparatus customary for this purpose, generally hollow modules such as Field tubes, tube coils, heat exchanger plates, etc. Alternatively, the reaction can also be effected in a cooled shell and tube reactor.

Preferably, the hydrogenation is effected in n series-connected hydrogenation reactors, where n is an integer of at least two, and wherein at least one reactor has a stream from the reaction zone conducted within an external circuit (external circulation stream, liquid circulation system, loop mode). Preferably, n is two or three.

Preferably, the hydrogenation is effected in n series-connected hydrogenation reactors, where n is preferably two or three, and the 1st to (n-1)th reactor has a stream from the reaction zone conducted within an external circuit.

Preferably, the hydrogenation is effected in n series-connected hydrogenation reactors, where n is preferably two or three, and wherein the reaction is conducted adiabatically in the nth reactor (the last reactor through which the reaction mixture to be hydrogenated flows).

Preferably, the hydrogenation is effected in n series-connected hydrogenation reactors, where n is preferably two or three, and wherein the nth reactor is operated in straight pass.

If a reactor is operated “in straight pass”, this shall be understood here and hereinafter to mean that a reactor is operated without recycling of the reaction product in the manner of a loop mode of operation. The mode of operation in straight pass does not fundamentally rule out backmixing internals and/or stirring units in the reactor.

When the reaction mixture hydrogenated in one of the reactors connected downstream of the first reactor (i.e. in the 2nd to nth reactor) has only such low proportions of hydrogenatable muconic acid that the exothermicity occurring in the reaction is insufficient to maintain the desired temperature in the reactor, heating of the reactor (or of individual reaction zones of the second reactor) may also be required. This can be effected analogously to the above-described removal of the heat of reaction by heating an external circulation stream or by internal heating. In a suitable embodiment, the temperature of a reactor can be controlled by using the heat of reaction from at least one of the upstream reactors.

In addition, the heat of reaction withdrawn from the reaction mixture can be used to heat the feed streams to the reactors. For this purpose, for example, the feed stream of the compound to be hydrogenated into the first reactor can be mixed at least partly with an external circulation stream of this reactor and then the combined streams can be conducted into the first reactor. In addition, in the case of m=2 to n reactors, the feed stream from the (m-1)th reactor can be mixed in the mth reactor with a circulation stream of the mth reactor, and the combined streams can then be conducted into the mth reactor. In addition, the feed stream of the compound to be hydrogenated and/or another feed stream can be heated with the aid of a heat exchanger which is operated with heat of hydrogenation withdrawn.

In a specific configuration of the process, a reactor cascade composed of n series-connected reactors is used, in which case the reaction is performed adiabatically in the nth reactor. In the context of the present invention, this term is used in the technical and not in the physicochemical sense. Thus, the reaction mixture generally experiences a temperature increase as it flows through the second reactor owing to the exothermic hydrogenation reaction. An adiabatic reaction regime is understood to mean a procedure in which the amount of heat released in the hydrogenation is absorbed by the reaction mixture in the reactor and no cooling by cooling apparatuses is employed. The heat of reaction is thus removed from the second reactor with the reaction mixture, apart from a residual fraction which is released to the environment by natural heat conduction and heat emission from the reactor. The nth reactor is preferably operated in straight pass.

In a preferred embodiment, the hydrogenation is effected using a two-stage reactor cascade, in which case the first hydrogenation reactor has a stream from the reaction zone conducted within an external circuit. In a specific embodiment of the process, a reactor cascade composed of two series-connected reactors is used, in which case the reaction is performed adiabatically in the second reactor.

In a further preferred embodiment, the hydrogenation is effected using a three-stage reactor cascade, in which case the first and second hydrogenation reactor have a stream from the reaction zone conducted within an external circuit. In a specific embodiment of the process, a reactor cascade composed of three series-connected reactors is used, in which case the reaction is performed adiabatically in the third reactor.

In one embodiment, additional mixing can be effected in at least one of the reactors used. Additional mixing is especially advantageous when the hydrogenation is effected with long residence times of the reaction mixture. Mixing can be effected, for example, using the streams introduced into the reactors, by introducing them into the particular reactors using suitable mixing devices, such as nozzles. Mixing can also be effected using streams from the particular reactor conducted within an external circuit.

To complete the hydrogenation, an output which still comprises hydrogenatable components is withdrawn from each of the first to (n-1)th reactors and is fed into the downstream hydrogenation reactor in each case. In a specific embodiment, the output is separated into a first and a second substream, in which case the first substream is fed back as a circulation stream to the reactor from which it has been withdrawn, and the second substream is fed to the downstream reactor. The output may comprise dissolved or gaseous fractions of hydrogen. In a specific embodiment, the output from the first to (n-1)th reactor is fed to a phase separation vessel and separated into a liquid phase and a gaseous phase, the liquid phase is separated into the first and the second substream, and the gas phase is fed separately at least partly to the downstream reactor. In an alternative embodiment, the output from the first to (n-1)th reactor is fed to a phase separation vessel and separated into a first liquid hydrogen-depleted substream and a second hydrogen-enriched substream. The first substream is then fed back as a circulation stream to the reactor from which it has been withdrawn, and the second substream is fed to the downstream reactor. In a further alternative embodiment, the second to nth reactor is charged with hydrogen not via a hydrogenous feed withdrawn from the upstream reactor but rather with fresh hydrogen via a separate feed line.

The above-described process variant is particularly advantageously suitable for control of the reaction temperature and of the heat transfer between reaction medium, delimiting apparatus walls and environment. A further means of controlling the heat balance consists in regulating the entry temperature of the compound to be hydrogenated. For instance, a lower temperature of the incoming feed generally leads to improved removal of the heat of hydrogenation. When the catalyst activity declines, the entry temperature can be selected at a higher level in order to achieve a higher reaction rate and thus to compensate for the decline in catalyst activity. Advantageously, it is generally possible in this way to prolong the service life of the hydrogenation catalyst used.

One-Stage or Two-Stage Hydrogenation

In a first preferred embodiment, the hydrogenation in step b) is effected without intermediate isolation of adipic acid or any ester of adipic acid.

In a second preferred embodiment, step b) of the process according to the invention comprises the following component steps:
b1) hydrogenating muconic acid or one of its esters in aqueous solution to adipic acid or one of its esters in the presence of a first hydrogenation catalyst, and
b2) hydrogenating the adipic acid or one of its esters in aqueous solution to hexane-1,6-diol in the presence of a second hydrogenation catalyst.

It is preferable here that the first catalyst is Raney cobalt and/or Raney nickel and/or Raney copper. It is further preferable here that the second catalyst, based on the total weight of the reduced catalyst, comprises at least 50% by weight of elements selected from the group consisting of rhenium, iron, ruthenium, cobalt, rhodium, iridium, nickel and copper. For hydrogenation of adipic acid, adipic monoesters and adipic diesters, it is especially preferable that the catalyst Hc2) comprises at least 50% by weight of elements selected from the group consisting of rhenium, ruthenium and cobalt. For hydrogenation of an adipic oligoester or polyester, it is especially preferable that the second catalyst comprises at least 50% by weight of copper.

The hydrogenation in step b1) is effected preferably at a temperature within the range from 50 to 160° C., more preferably 60 to 150° C., most preferably 70 to 140° C. Within this temperature range, preferably more than 50%, more preferably more than 70%, most preferably more than 90%, of the carbon-carbon double bonds present in the muconic acid are hydrogenated.

The hydrogenation in step b) is effected preferably at a temperature in the range from 160 to 240° C., more preferably 170 to 230° C., most preferably 170 to 220° C. This also hydrogenates the as yet unhydrogenated carbon-carbon double bonds and the carboxyl groups.

Step b1) can be conducted, for example, in a first loop reactor and step b2) in a second loop reactor. The conversion in step b2) can be completed in a downstream tubular reactor. However, it is also possible to manage with one loop reactor when two temperature zones are provided therein. In this case too, a tubular reactor in a straight pass follows downstream. The hydrogenations can be effected in liquid phase mode or trickle mode.

Workup of the hexane-1,6-diol

The reaction output obtained in the hydrogenation of muconic acid in water as solvent is an aqueous hexane-1,6-diol solution. After the cooling and decompression of the hydrogenation output, the water is removed by distillation in step c), and hexane-1,6-diol can be obtained in high purity (>97%).

If the muconic acid hydrogenation is conducted, for example, in methanol as a solvent, a portion of the muconic acid is converted in situ to monomethyl muconate and dimethyl muconate. The hydrogenation output is a solution of hexane-1,6-diol in a mixture of methanol and water. By distillation, methanol and water are separated from hexane-1,6-diol. Methanol is preferably separated from water and recycled into the hydrogenation. Water is discharged.

If n-butanol or i-butanol is used as a solvent in the muconic acid hydrogenation, a liquid biphasic mixture is obtained after the cooling and decompression of the hydrogenation output. The aqueous phase is separated from the organic phase by phase separation. The organic phase is distilled. Butanol is removed as the top product and preferably recycled into the muconic acid hydrogenation. Hexane-1,6-diol can, if necessary, be purified further by distillation.

If muconic diesters are used for hydrogenation, substantially anhydrous solutions of hexane-1,6-diol are obtained, which can be worked up by distillation to give pure hexane-1,6-diol. The alcohols obtained are preferably recycled into the esterification stage.

Hydrogenation of muconic oligo- and polyesters comprising hexane-1,6-diol as the diol component gives a hydrogenation output consisting very predominantly of hexane-1,6-diol.

The invention is illustrated in detail by the nonlimiting examples which follow.

### EXAMPLES

There follows a description of the two-stage synthesis of hexane-1,6-diol in one embodiment of the process according to the invention.

### Example 1

Preparation of Muconic Acid

cis,cis-Muconic acid was prepared by the method in K. M. Draths, J. W. Frost, J. Am. Chem. Soc., 116 (1994), pages 399-400, biocatalytically from D-glucose by means of the Escherichia coli mutant AB2834/pKD136/pKD8.243A/pKD8.292.

### Example 2

Preparation of Adipic Acid

A 250 mL stirred autoclave was charged with a suspension of 24 g of the cis,cis-muconic acid and 1 g of Raney Ni in 56 g of water, hydrogen was injected to 3 MPa and the autoclave was heated to 80° C. On attainment of the temperature of 80° C., the pressure was increased to 10 MPa and a sufficient amount of further hydrogen was metered in to keep the pressure constant. After a reaction time of 12 h, the autoclave was cooled to a temperature of 60° C. and decompressed to standard pressure, and the catalyst was filtered out of the solution. Thereafter, the mixture was cooled gradually to 20° C., in the course of which adipic acid crystallized out as a white solid. In the solution, as well as adipic acid, it was still possible to detect lactone (V). The yield of adipic acid was 95% and that of lactone (V) 5%.

Preparation of hexane-1,6-diol (Continuous Hydrogenation)

15 g/h of a mixture of 33% of the adipic acid and 67% water were hydrogenated at a feed temperature of 70° C. in a 30 mL tubular reactor in which 20 mL of catalyst (66% CoO, 20% CuO, 7.3% Mn₃O₄, 3.6% MoO₃, 0.1% Na₂O, 3% H₃PO₄, preparation according to DE 23 21 101 A; 4 mm extrudates; activation with hydrogen up to 300° C.) were present, in trickle mode at a temperature of 230° C. and a pressure of 25 MPa. The reactor output was separated from excess hydrogen in a separator (offgas rate 2 L/h) and passed partly through a pump as circulation stream back to the head of the reactor, where it is combined with the feed stream (feed:circulation=1:10), and partly into an output vessel. The outputs were analyzed by gas chromatography (% by weight, method with internal standard). The yield of hexane-1,6-diol was 94%; the yield of adipic acid was 98.5%. As further products, 3% 6-hydroxycaproic acid, 1% hexane-1,6-diol 6-hydroxycaproate and 1% hexanol were present.

1.-22. (canceled)

23. A process for preparing hexane-1,6-diol which comprises
a) Providing a muconic acid starting material selected from muconic acid, esters of muconic acid, lactones of muconic acid and mixtures thereof,
b) Subjecting the muconic acid starting material to a reaction with hydrogen in the presence of at least one hydrogenation catalyst to hexane-1,6-diol, and
wherein the hydrogenation in step b) is effected without intermediate isolation of adipic acid or any ester of adipic acid, or
wherein step b) comprises the following sub-steps:
b1) hydrogenating muconic acid or one of its esters in aqueous solution to adipic acid in the presence of a first hydrogenation catalyst, and
b2) hydrogenating the adipic acid in aqueous solution to hexane-1,6-diol in the presence of a second hydrogenation catalyst,
and
c) the output from the hydrogenation in step b) is subjected to a distillative separation to obtain hexane-1,6-diol.

24. The process according to claim 23, wherein the muconic acid starting material is provided in step a), in which the muconic acid originates from a renewable source, and is prepared by biocatalytic synthesis from at least one renewable raw material.

25. The process according to claim 23, wherein the muconic acid used in step a) has a ¹⁴C-to-¹²C isotope ratio in the range from 0.5×10⁻¹² to 5×10⁻¹².

26. The process according to claim 23, wherein the hydrogenation in step b) is effected using a muconic acid starting material selected from the group consisting of muconic acid, muconic monoesters, muconic diesters, poly(muconic esters) and mixtures thereof.

27. The process according to claim 23, wherein the hydrogenation in step b) is effected using a muconic acid starting material selected from the lactones (III), (IV) and (V) and mixtures thereof:

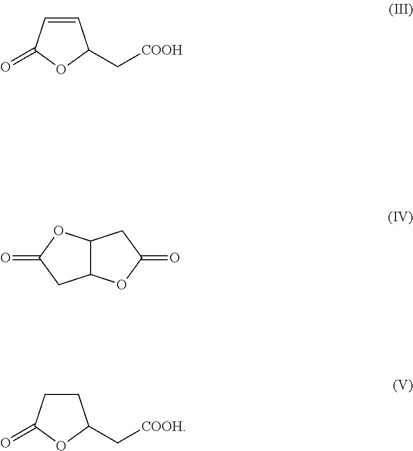 [SMILES: O=C(O)CC1C=CC(=O)O1 ; O=C1CC2OC(=O)CC2O1 ; O=C(O)CC1CCC(=O)O1]

28. The process according to claim 23, wherein the hydrogenation in step b) is effected in the liquid phase in the presence of a solvent selected from the group consisting of water, aliphatic C₁ to C₅ alcohols, aliphatic C₂ to C₆ diols, ethers and mixtures thereof.

29. The process according to claim 23, wherein the hydrogenation in step b) is effected in the liquid phase in the presence of water as the sole solvent.

30. The process according to claim 23, wherein the hydrogenation in step b) is effected in the gas phase using for the hydrogenation a muconic diester selected from compounds of the general formula (II):
R¹OOC—CH═CH—CH═CH—COOR² (II)
in which the R¹ and R² radicals are each independently straight-chain or branched C₁-C₅-alkyl.

31. The process according to claim 23, wherein the hydrogenation catalyst used in step b) is a heterogeneous transition metal catalyst.

32. The process according to claim 23, wherein the hydrogenation in step b) is effected in the liquid phase in the presence of water as the sole solvent, wherein the hydrogenation catalyst used is a heterogeneous transition metal catalyst.

33. The process according to claim 23, wherein
in step b) a muconic acid starting material is used, selected from the group consisting of muconic acid, muconic monoesters and mixtures thereof, and a heterogeneous hydrogenation catalyst is used, comprising at least 50% by weight of cobalt, ruthenium or rhenium, based on the total weight of the reduced catalyst, or
in step b) a muconic acid starting material is used, selected from the group consisting of muconic diesters, poly(muconic esters) and mixtures thereof, and a heterogeneous hydrogenation catalyst is used, comprising at least 50% by weight of copper, based on the total weight of the reduced catalyst.

34. The process according to claim 23, wherein the hydrogenation in step b) is effected at a temperature within the range from 50 to 300° C.

35. The process according to claim 23, wherein the hydrogenation in step b) is effected at a partial hydrogen pressure within a range from 100 to 300 bar.

36. The process according to claim 23, wherein the hydrogenation in step b) comprises the following component steps:
b1) hydrogenating muconic acid or one of its esters in water as the sole solvent to adipic acid in the presence of a first heterogeneous hydrogenation catalyst, and
b2) hydrogenating the adipic acid obtained in step b1) in water as the sole solvent to hexane-1,6-diol in the presence of a second heterogeneous hydrogenation catalyst,
the hydrogenation being effected continuously at least in step b2).

37. The process according to claim 23, wherein the first hydrogenation catalyst is Raney cobalt and/or Raney nickel.

38. The process according to claim 23, wherein the second catalyst, based on the total weight of the reduced catalyst, comprises at least 50% by weight of elements selected from the group consisting of rhenium, iron, ruthenium, cobalt, rhodium, iridium, nickel and copper.

39. The process according to claim 23, wherein the hydrogenation in step b1) is effected at a temperature within the range from 50 to 160° C. and the hydrogenation in step b2) is effected at a temperature within the range from 160 to 240° C.

40. The process according to claim 23, wherein adipic acid-containing water which is obtained in the isolation of the second catalyst on completion of step b2) is used as solvent in step b1).

41. The process according to claim 23, wherein the hydrogenation is conducted in n series-connected hydrogenation reactors, where n is an integer of at least two, and wherein the 1st to (n-1)th reactor has a stream from the reaction zone which is conducted within an external circuit and the hydrogenation in the nth reactor is conducted adiabatically.

42. The process according to claim 23, wherein the poly(muconic ester) is of the general formula (VI) 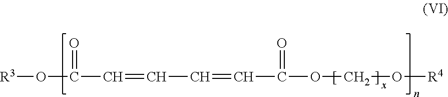
in which
x is an integer from 2 to 6,
n is an integer from 1 to 100,
R³ is H, straight-chain or branched C₁-C₅-alkyl or a HO—(CH₂)ₓ— group,
R⁴ is H or a —C(═O)—CH═CH—CH⊚CH—COOR⁵ group in which R⁵ is H or straight-chain or branched C₁-C₅-alkyl,
with the proviso that, when n=1, either R³ is H and R⁴ is —C(═O)—CH═CH—CH═CH—COOR⁵ or R³ is a HO—(CH₂)ₓ— group and R⁴ is H.

43. Hexane-1,6-diol having a C¹⁴/C¹² isotope ratio in the range from 0.5×10⁻¹² to 5×10⁻¹².

44. Hexane-1,6-diol preparable proceeding from muconic acid synthesized biocatalytically from at least one renewable raw material.
